(19) United States
(12) Patent Application Publication
(10) Pub. No.: US 2018/0363211 A1
(43) Pub. Date: Dec. 20, 2018

(54) WALL-MOUNTED WASHING MACHINE

(71) Applicant: Dongbu Daewoo Electronics Corporation, Seoul (KR)

(72) Inventor: Sang Ki HAN, Seoul (KR)

(21) Appl. No.: 15/872,857

(22) Filed: Jan. 16, 2018

(30) Foreign Application Priority Data
Jun. 14, 2017 (KR) 10-2017-0074713

(51) Int. Cl. D06F 39/00; D06F 39/12; D06F 37/04; D06F 25/00; A61L 2/10

(52) U.S. Cl. CPC D06F 39/004; D06F 39/12; A61L 2/10; D06F 25/00; D06F 37/04

(57) ABSTRACT

A wall-mounted washing machine includes a cabinet, a tub installed inside the cabinet and having an open front surface, a tub front cover coupled to the front surface of the tub and having an opening corresponding to the open front surface of the tub, a drum rotatably installed inside the tub, and a gasket fitted to a rim of the opening of the tub front cover. The washing machine further includes a sterilization unit coupled to a front surface of the tub front cover. At least a part of the sterilization unit is configured to penetrate a side wall around the opening of the tub front cover. An insertion portion into which the sterilization unit is inserted is provided in a front portion of the gasket, and the sterilization unit is in the insertion portion in a sealed state.

### CROSS-REFERENCE TO RELATED APPLICATION

This application is based on and claims priority to Korean Patent Application No. 10-2017-0074713, filed on Jun. 14, 2017, the disclosure of which is incorporated herein in its entirety by reference for all purposes.

### TECHNICAL FIELD

Embodiments according to the present disclosure relate to a wall-mounted washing machine.

### BACKGROUND

In general, a wall-mounted washing machine is smaller than a typical drum-type washing machine and can be installed on a wall surface in a narrow space. The wall-mounted washing machine is designed to easily wash objects such as baby clothes, undergarments or the like, which may have to be frequently washed.

The mounting surface includes a cabinet, a tub disposed within the cabinet and configured to store washing water, a drum rotatably installed in the tub and configured to accommodate objects being washed, a drive unit configured to supply power to the drum, a water supply device configured to supply the washing water into the tub, and a water drain device configured to drain the washing water from the tub to the outside of the cabinet.

For example, if a washing operation is started after a user puts objects into the drum, the washing water is supplied into the tub and the drum by the operation of the water supply device. The drum is rotated by the drive unit. Washing is completed through a washing process and a dewatering (water removal) process. At the end of washing, the washing water contained in the tub and the drum is drained to the outside of the cabinet by the water drain device.

If such a washing operation is repeated, foreign materials such as dirt and the like or contaminants such as detergent residue and the like may remain and accumulate inside the drum. In particular, the interior of the drum is always exposed to a humid environment. Therefore, various bacteria may be present in the foreign materials or contaminants remaining inside the drum. As a result, there is a problem in that objects placed in the drum may be contaminated.

Accordingly, a need exists for a wall-mounted washing machine capable of sterilizing and disinfecting a drum and objects placed in the drum for washing.

Korean Patent Application Publication No. 10-2013-0064623 (published on Jun. 18, 2013) is an example of the prior art.

### SUMMARY

Embodiments according to the present disclosure provide a wall-mounted washing machine capable of sterilizing and disinfecting a drum and objects to be washed or being washed.

In embodiments, a wall-mounted washing machine includes: a cabinet; a tub installed inside the cabinet and having an open front surface; a tub front cover coupled to the front surface of the tub and having an opening corresponding to the open front surface of the tub; a drum rotatably installed inside the tub; a gasket fitted to a rim of the opening of the tub front cover; and a sterilization unit coupled to a front surface of the tub front cover. At least a part of the sterilization unit is configured to penetrate a side wall around the opening of the tub front cover. An insertion portion into which the sterilization unit is inserted is in a front portion of the gasket, and the sterilization unit is in the insertion portion in a sealed state.

In embodiments, the sterilization unit includes a fastening portion fastened to a front surface of the tub front cover, a case portion connected to the fastening portion and disposed in the insertion portion of the gasket, an ultraviolet light source unit disposed inside the case portion and configured to emit ultraviolet light, and a cover portion connected to the case portion and configured to cover the ultraviolet light source unit. At least a part of the cover portion is exposed through an exposure hole formed in the insertion portion of the gasket.

In embodiments, the ultraviolet light source unit includes a printed circuit board disposed inside the case portion, a support means disposed inside the case portion and configured to support the printed circuit board, and an ultraviolet light emitting diode mounted on the printed circuit board.

In embodiments, one surface of the case portion to which the cover portion is coupled is inclined toward the inside of the drum.

In embodiments, the sterilization unit is disposed in a front upper portion of the tub front cover.

In embodiments, the washing machine further includes a drying device coupled to an upper outer surface of the tub and configured to dry objects that were washed. In embodiments, the drying device includes a fan coupled to an upper portion of the tub and configured to circulate air, a hot air supply duct connected to the fan and configured to supply the air into the tub, a drying heater provided inside the hot air supply duct and configured to heat the air introduced into the hot air supply duct, and a hot air discharge duct connected to the tub and configured to guide the air passing through the tub toward the fan.

In embodiments, the sterilization unit is disposed closer to the fan than the hot air supply duct.

In other embodiments, a wall-mounted washing machine includes: a tub having an open front surface; a tub front cover coupled to the front surface of the tub and having an opening corresponding to the open front surface of the tub; a drum rotatably installed inside the tub; a gasket fitted to a rim of the opening of the tub front cover; and a sterilization unit coupled to a front surface of the tub front cover. Further, at least a part of the sterilization unit is configured to penetrate a side wall around the opening of the tub front cover and fixed by the gasket in a sealed state.

In embodiments, the sterilization unit includes a fastening portion fastened to a front surface of the tub front cover, a case portion connected to the fastening portion and disposed in an insertion portion of the gasket, an ultraviolet light source unit disposed inside the case portion and configured to emit ultraviolet light, and a cover portion connected to the case portion and configured to cover the ultraviolet light source unit. At least a part of the cover portion is exposed through an exposure hole formed in the insertion portion of the gasket.

In embodiments, the ultraviolet light source unit includes a printed circuit board disposed inside the case portion, a support means disposed inside the case portion and configured to support the printed circuit board, and an ultraviolet light emitting diode mounted on the printed circuit board.

In embodiments, one surface of the case portion to which the cover portion is coupled is inclined toward the inside of the drum.

In embodiments, the sterilization unit is disposed in a front upper portion of the tub front cover.

In embodiments, the washing machine further includes a drying device coupled to an upper outer surface of the tub and configured to dry objects that were washed. Further, in these embodiments, the drying device includes a fan coupled to an upper portion of the tub and configured to circulate air, a hot air supply duct connected to the fan and configured to supply the air into the tub, a drying heater provided inside the hot air supply duct and configured to heat the air introduced into the hot air supply duct, and a hot air discharge duct connected to the tub and configured to guide the air passing through the tub toward the fan.

In embodiments, the sterilization unit is disposed closer to the fan than the hot air supply duct.

A wall-mounted washing machine in embodiments according to the present disclosure sterilizes and disinfects a drum and objects in the drum.

### BRIEF DESCRIPTION OF THE DRAWINGS

FIG. 1 is a front perspective view of a wall-mounted washing machine in an embodiment according to the present disclosure.

FIG. 2 is a rear perspective view of the wall-mounted washing machine shown in FIG. 1 in an embodiment according to the present disclosure.

FIG. 3 is an exploded perspective view of the wall-mounted washing machine shown in FIG. 1 in an embodiment according to the present disclosure.

FIG. 4 is a perspective view showing a rear panel and a tub of the wall-mounted washing machine shown in FIG. 1 in an embodiment according to the present disclosure.

FIG. 5 is a perspective view showing a tub front cover and a gasket of the wall-mounted washing machine shown in FIG. 1 in an embodiment according to the present disclosure.

FIG. 6 is a sectional view taken along line VI-VI in FIG. 5 in an embodiment according to the present disclosure.

FIG. 7 is a perspective view showing a tub front cover of the wall-mounted washing machine shown in FIG. 1 in an embodiment according to the present disclosure.

FIG. 8 is a perspective view showing a gasket of the wall-mounted washing machine shown in FIG. 1 in an embodiment according to the present disclosure.

FIG. 9 is a perspective view showing a sterilization unit of the wall-mounted washing machine shown in FIG. 1 in an embodiment according to the present disclosure.

FIG. 10 is a sectional view taken along line X-X in FIG. 9 in an embodiment according to the present disclosure.

### DETAILED DESCRIPTION

In the following detailed description, reference is made to the accompanying drawings. The illustrative embodiments described in the detailed description, drawings, and claims are not meant to be limiting. Other embodiments may be utilized, and other changes may be made, without departing from the spirit or scope of the subject matter presented here.

One or more example embodiments according to the present disclosure will be described more fully hereinafter with reference to the accompanying drawings, in which one or more embodiments of the disclosure can be easily determined by those skilled in the art. As those skilled in the art will realize, the described example embodiments may be modified in various different ways, all without departing from the spirit or scope of the present disclosure, which is not limited to the embodiments described herein.

It is noted that the drawings are schematic and are not necessarily illustrated to scale. Relative sizes and proportions of parts in the drawings may be exaggerated or reduced in size, and a predetermined size if indicated is merely an example and not limiting. The same reference numerals designate the same structures, elements, or parts illustrated in two or more drawings in order to exhibit similar characteristics.

The drawings of the present disclosure illustrate example embodiments according to the present disclosure in more detail. As a result, various modifications of the drawings are expected. Accordingly, the example embodiments are not limited to a specific form of the illustrated region, and for example, may include modification of form due to manufacturing.

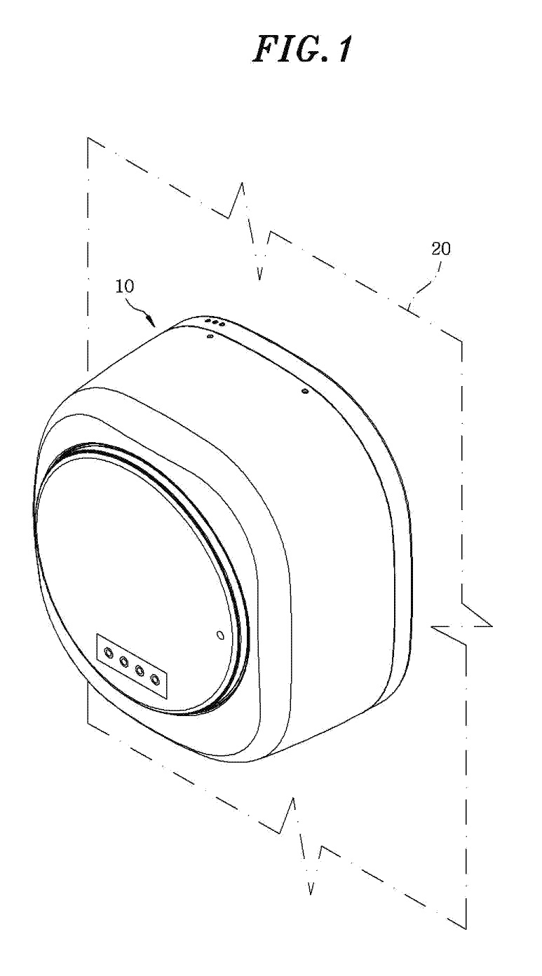

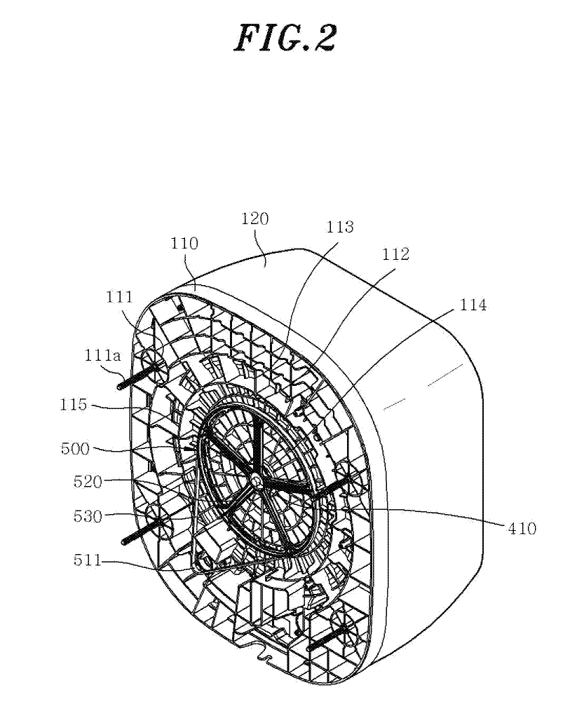

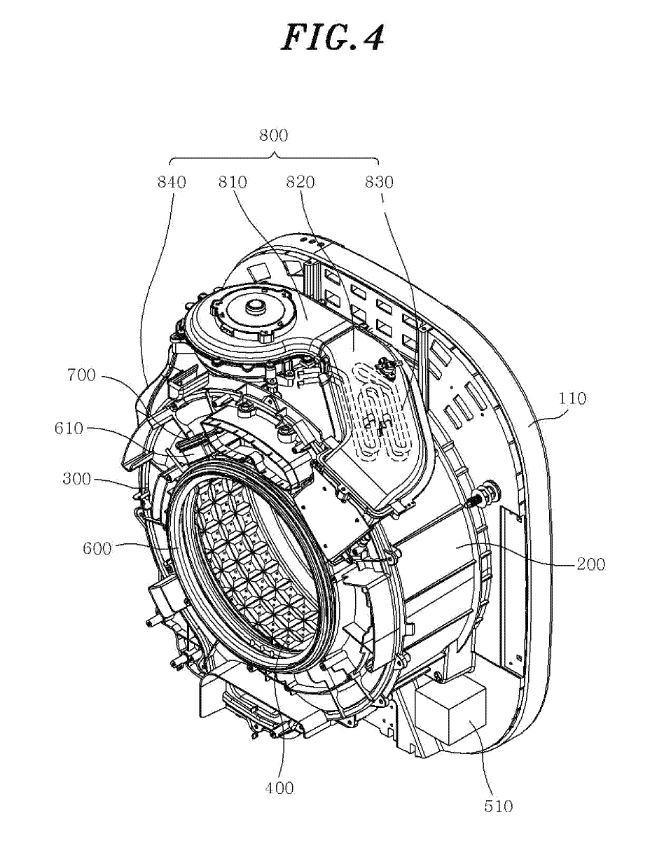

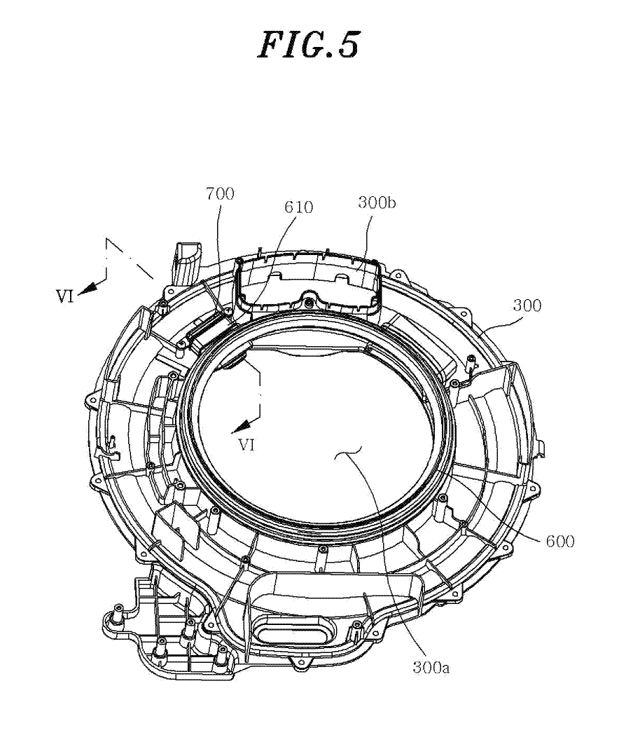

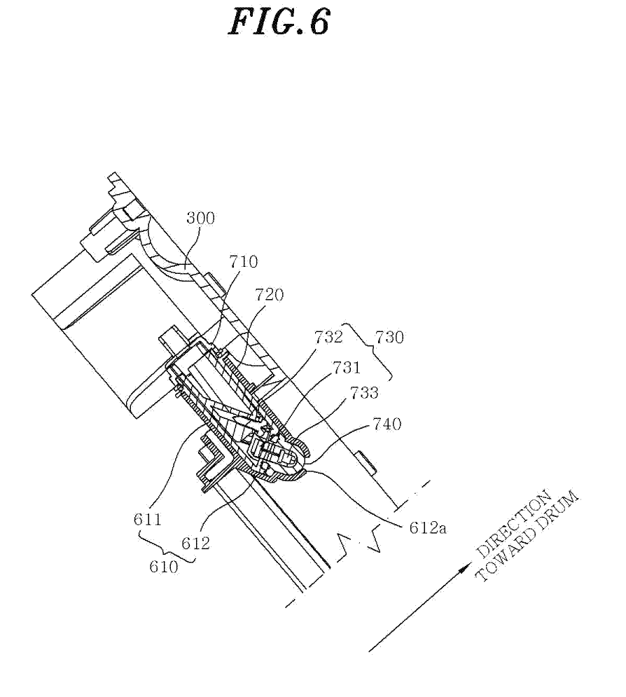

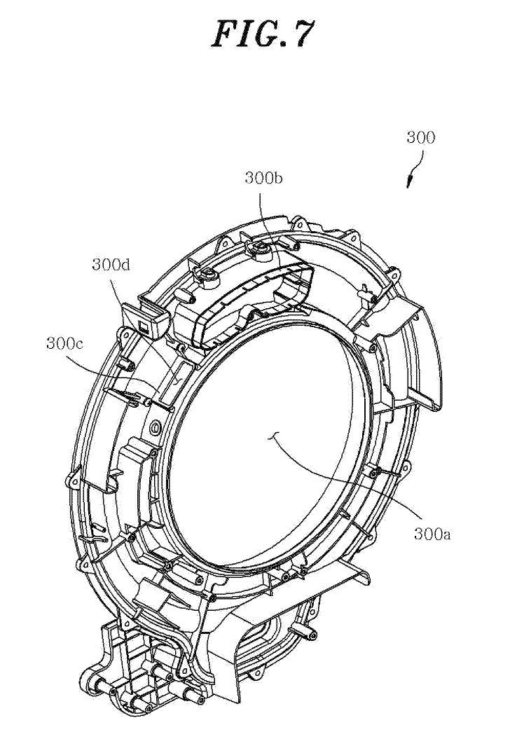

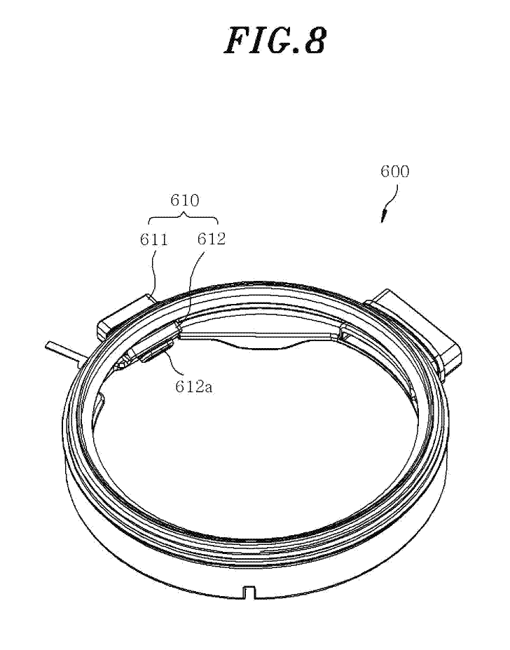

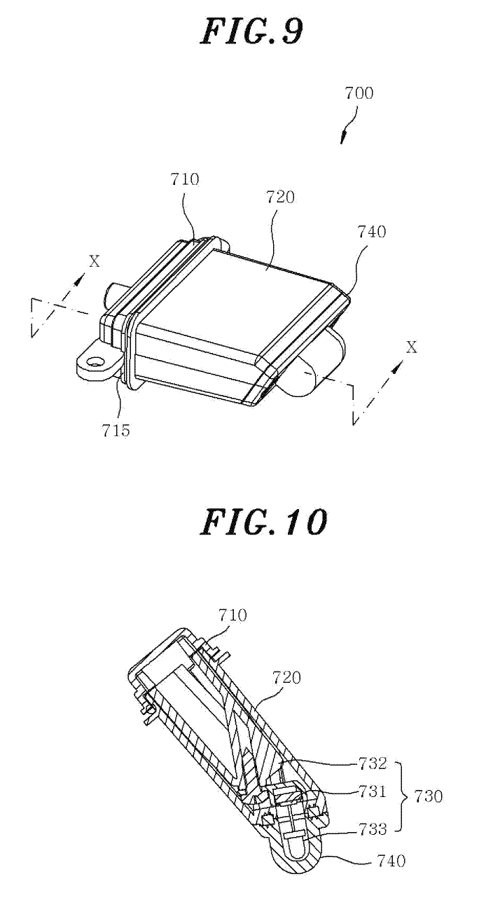

A wall-mounted washing machine 10 according to an embodiment of the present disclosure will now be described with reference to FIGS. 1 to 10.

Referring to FIGS. 1 to 10, the wall-mounted washing machine 10 according to an embodiment of the present disclosure may be a drum-type washing machine installed on a wall surface 20 to easily wash objects such as baby clothes, undergarments or the like, which may have to be frequently washed, and is also capable of drying the objects that have been washed. The wall-mounted washing machine 10 may include, for example, a cabinet 100, a tub 200, a tub front cover 300, a drum 400, a drive unit 500, a gasket 600, a sterilization unit 700, and a drying device 800.

The cabinet 100, which forms an outer shell of the wall-mounted washing machine 10, may include a rear panel 110, a cover portion 120, and a front panel 130.

The rear panel 110 is mounted on the wall surface 20 by a fastening member 111a. The tub 200 may be integrally formed with the front surface of the rear panel 110. For example, the rear panel 110 may have a rectangular shape with rounded corners. However, this is an example, and the present disclosure is not so limited. For example, the rear panel 110 may instead be a circular shape, an elliptical shape, or the like.

On the rear surface of the rear panel 110, there may be through-hole portions 111, first reinforcing ribs 112, second reinforcing ribs 113, a connecting portion 114, and a mounting recess portion 115.

The through-hole portions 111, into which fastening members 111a for mounting the rear panel 110 to the wall surface 20 are inserted, may be respectively formed in the corners of the rear surface of the rear panel 110. The fastening members 111a, inserted into the through-hole portions 111, may be coupled to the wall surface 20.

The first reinforcing ribs 112 may be radially formed in a plural number about the central portion of the rear surface of the rear panel 110 to prevent the rear panel 110 from being damaged by the vibration generated during the rotation of the drum 400. The first reinforcing ribs 112 may be connected to one another by the second reinforcing ribs 113.

The connecting portion 114 is provided in the central portion of the rear surface of the rear panel 110 and may connect the drum 400 and the drive unit 500. The driving shaft 410 of the drum 400 (described below) may be rotatably installed in the connecting portion 114. In that case, the drum 400 is installed in one end portion of the driving shaft 410 of the drum 400 installed in the connecting portion 114, and the driving wheel 520 of the drive unit 500 (described below) is installed in the other end portion of the driving shaft 410 of the drum 400. As a result, the power supplied from the drive unit 500 may be transmitted to the drum 400.

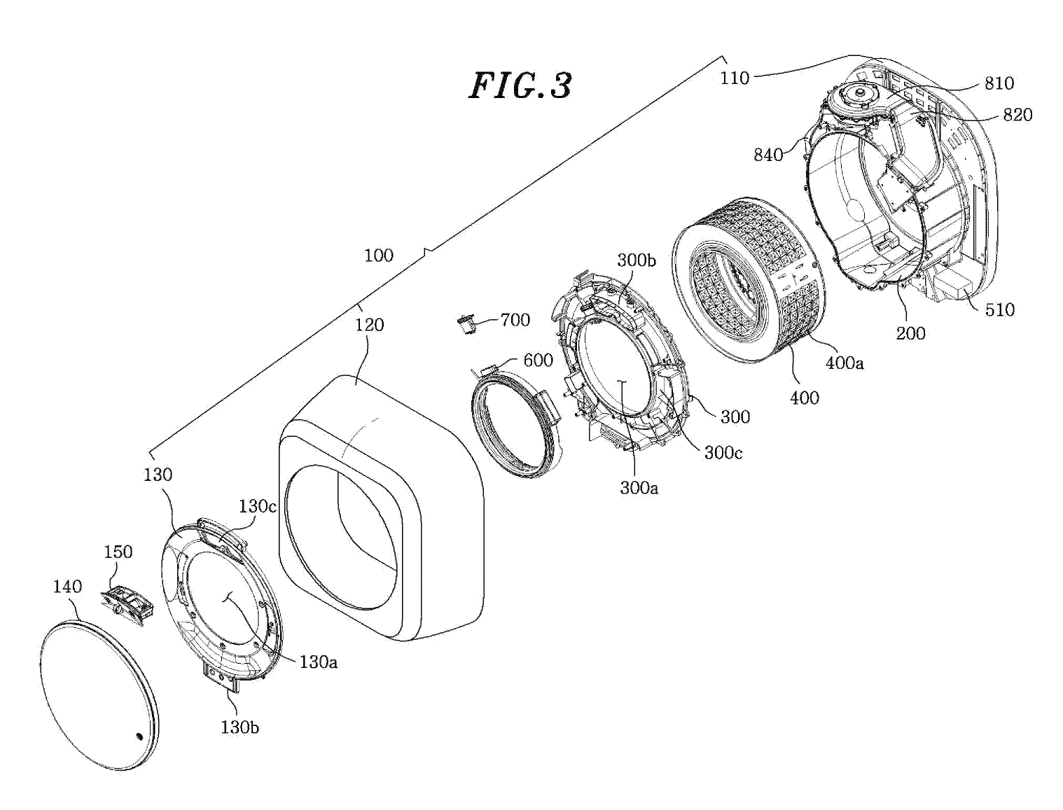

The driving wheel 520 of the drive unit 500 is mounted to the mounting recess portion 115. The mounting recess portion 115 may be recessed frontward from the rear surface of the rear panel 110 to prevent the driving wheel 520 from interfering with the rear panel 110 and the wall surface 20. As used herein, the term “frontward” refers to the direction extending from the rear panel 110 toward the tub 200 on the basis of FIG. 3, and the term “rearward” refers to the direction extending from the tub 200 toward the rear panel 110 on the basis of FIG. 3.

The cover portion 120 is coupled to the rear panel 110 and is configured to surround the outer circumferential surface of the tub 200. The cover portion 120 may have a structure in which a part of the front surface and the rear surface are open. As an example, the cover portion 120 may be formed in a rectangular tube shape with round corners so that its perimeter gradually decreases frontward.

The front panel 130 may be installed on the open front surface of the cover portion 120. An opening 130a may be formed in the central portion of the front panel 130. An operation unit 130b, for controlling the wall-mounted washing machine 10, may be disposed in the front lower portion of the front panel 130 surrounding the opening 130a.

To open and close the opening 130a of the front panel 130, a door 140 may be coupled to the front panel 130. In this case, the door 140 may be an electronic door which is opened and closed in a one-touch manner by the operation of the operation unit 130b. Alternatively, the door 140 may be configured so that a user can directly open and close the door 140 using a handle (not shown) of the door 140.

Meanwhile, a detergent box installation portion 130c may be disposed in the front upper portion of the front panel 130 surrounding the opening 130a. A detergent box 150, for storing a liquid detergent and a fabric softener, is removably installed in the detergent box installation portion 130c. As an example, the detergent box installation portion 130c may be formed in a shape corresponding to the shape of the detergent box 150.

The tub 200 stores washing water. The tub 200 may receive the washing water from the water supply device (not shown) and may discharge the washing water to the outside of the cabinet 100 through the water drain device (not shown). To this end, the water supply device may be connected to the upper portion of the tub 200, and the water drain device may be connected to the lower portion of the tub 200.

The tub 200 may protrude frontward from the front surface of the rear panel 110. As an example, the tub 200 may have a cylindrical shape so that the diameter of its side wall gradually decreases frontward. In addition, the tub 200 may be made of substantially the same material as the rear panel 110, e.g., a synthetic resin, and may be manufactured through an insert injection molding process performed once. However, this is an example. The tub 200 may be formed as a separate member and may be coupled to the front surface of the rear panel 110.

The tub front cover 300 may be on the front surface of the tub 200 and may define an internal space in which the drum 400 is disposed. In this case, an opening 300a may be dispose in the central portion of the tub front cover 300. The gasket 600 may be fitted to the rim portion that defines the opening 300a. To increase the coupling force with the gasket 600, the rim portion that defines the opening 300a may have a step-like shape.

A detergent box insertion portion 300b may be on the front surface of the tub front cover 300 that surrounds the opening 300a. The detergent box 150 is inserted into the detergent box insertion portion 300b. The detergent box insertion portion 300b may protrude frontward from the front surface of the tub front cover 300. In this case, the side wall 300c that defines the opening 300a protrudes frontward from the front surface of the tub front cover 300. In embodiments, a sterilization unit 700 is disposed in an insertion hole 300d in the side wall 300c.

The drum 400, which accommodates the objects being washed, may be rotatably installed inside the tub 200. The drum 400 may receive the washing water stored inside the tub 200 through the washing water introduction holes 400a formed on the outer circumferential surface of the drum 400.

The driving shaft 410 may be connected to the drum 400. The driving shaft 410 may be connected to the drive unit 500 disposed on the rear surface of the rear panel 110 and may receive power from the drive unit 500.

The drive unit 500 is disposed on the rear surface of the rear panel 110 and is configured to supply power for rotating the drum 400. The drive unit 500 may include a driving motor 510, a driving wheel 520, and a belt 530.

The driving motor 510, which supplies power for rotating the drum 400, may be on the front surface of the rear panel 110. In this case, the rotating shaft 511 of the driving motor 510 may penetrate the rear panel 110 and may extend toward the rear surface of the rear panel 110.

The driving wheel 520, which is rotated by the power of the driving motor 510 transferred via the belt 530, may be inside the mounting recess portion 115 formed on the rear surface of the rear panel 110 and may be connected to the driving shaft 410 of the drum 400.

The belt 530, which is configured to transmit the power of the driving motor 510 to the driving wheel 520, may operatively connect the rotating shaft 511 of the driving motor 510 and the driving shaft 410 of the drum 400. Thus, the power of the driving motor 510 is transmitted to the driving wheel 520 by the belt 530. The driving wheel 620 is rotated without interfering with the rear panel 110 and the wall surface 20, whereby the drum 400 connected to the driving shaft 410 of the drum 400 may be rotated.

In embodiments, the gasket 600 is fitted to the rim defining the opening 300a of the tub front cover 300, thereby sealing a gap between the cabinet 100 and the tub 200 and also sealing the sterilization unit 700. In embodiments, an insertion portion 610 that can accommodate the sterilization unit 700 is disposed in the front upper portion of the gasket 600.

Specifically, the insertion portion 610 may include an outer insertion portion 611 and an inner insertion portion 612.

The outer insertion portion 611 protrudes radially outward of the tub front cover 300 from at least a part of the outer circumferential surface of the gasket 600. In embodiments, part of a case portion 720 of the sterilization unit 700 (described below) is inserted into and sealed by the outer insertion portion 611.

The inner insertion portion 612 may be formed to protrude radially inward of the tub front cover 300 from the inner circumferential surface of the gasket 600 corresponding to the outer circumferential surface of the gasket 600 on which the outer insertion portion 611 is formed.

In embodiments, the remainder of the case portion 720 of the sterilization unit 700 (described below) and a cover portion 740 are inserted into and sealed by the inner insertion portion 612. In this case, the inner insertion portion 612 may be formed so as to be inclined at a predetermined angle toward the inside of the drum 400.

The inner insertion portion 612 may expose at least a part of the cover portion 740 to the outside. To this end, an exposure hole 612a may be formed on the outer surface of the inner insertion portion 612. As an example, the exposure hole 612a may be formed in an elliptical shape so as to penetrate the outer surface of the inner insertion portion 612. The rim that defines the exposure hole 612a may protrude from the outer surface of the inner insertion portion 612.

The sterilization unit 700 sterilizes and disinfects the interior of the drum 400 and the objects inside the drum 400. In embodiments, the sterilization unit 700 is fastened to the front surface of the tub front cover 300 in a state in which the sterilization unit 700 is inserted and accommodated in the insertion portion 610 of the gasket 600.

In embodiments, the sterilization unit 700 includes a fastening portion 710, a case portion 720, an ultraviolet light source unit 730, and a cover portion 740. A method of fastening the sterilization unit 700 will now be briefly described. The gasket 600 is first fitted to the rim that defines the opening 300a of the tub front cover 300. The case portion 720 and the cover portion 740 of the sterilization unit 700 are inserted and accommodated in the insertion portion 610 of the gasket 600. Thereafter, the fastening portion 710 of the sterilization unit 700 is fastened to the tub front cover 300.

In embodiments, one end of the fastening portion 710 is fastened to the front surface of the tub front cover 300. At least a part of the case portion 720 is inserted into the other end of the fastening portion 710. In embodiments, an O-ring 715 may be coupled to the outer surface of the fastening portion 710 into which at least a part of the case portion 720 is inserted. As a result, the gap between the fastening portion 710 and the case portion 720 is tightly sealed by the O-ring 715. Furthermore, in embodiments, the fastening portion 710 is fastened to a support means 732 of the ultraviolet light source unit 730, which is provided inside the case portion 720, via a separate fastening member.

In embodiments, the case portion 720, which is inserted and accommodated in the insertion portion 610 of the gasket 600, is connected to the fastening portion 710. In embodiments, the ultraviolet light source unit 730 is inside the case portion 720. In this case, the surface of the case portion 720 opposite to the side surface connected to the fastening portion 710 has a slanted surface inclined at a predetermined angle toward the inside of the drum 400.

The ultraviolet light source unit 730 can radiate light in the ultraviolet range, e.g., light having a wavelength in a range of 250 nm to 260 nm, into the drum 400, to decompose contaminants such as an organic contaminant, bacteria, mold and the like, which may be presented in the interior of the drum 400 and the objects to be washed. This makes it possible to sterilize and disinfect the interior of the drum 400 and the objects inside the drum 400.

To this end, the ultraviolet light source unit 730 is disposed inside the case portion 720. In embodiments, the ultraviolet light source unit 730 includes a printed circuit board 731, a support means 732 fastened to the printed circuit board 731 and configured to support the printed circuit board 731, and an ultraviolet light emitting diode (LED) 733 mounted on the printed circuit board 731 and configured to emit ultraviolet light using electric power supplied from an external power source.

In embodiments, the side surface of the support means 732 fastened to the printed circuit board 731 has a slanted surface having substantially the same inclination angle as the slanted surface of the case portion 720. Thus, the ultraviolet light emitting diode 733 mounted on the printed circuit board 731 fastened to the slant surface of the support means 732 can emit ultraviolet light toward the inside of the drum 400.

Instead of the ultraviolet light emitting diode 733, the ultraviolet light source unit 730 may include an ultraviolet lamp, for example.

In embodiments, the cover portion 740 is fastened to the case portion 720 via a separate fastening member to cover the ultraviolet light source unit 730. For example, the cover portion 740 has a dome shape in which the part of the cover portion 740 corresponding to the ultraviolet light emitting diode 733 of the ultraviolet light source unit 730 protrudes more outward than the remaining part of the cover portion 740. The cover portion 740 may be made of a material through which ultraviolet light can be transmitted.

The ultraviolet light source unit 730 has electronic parts. For that reason, when heat having a predetermined temperature or higher is applied to the ultraviolet light source unit 730, there is a possibility that the ultraviolet light source unit 730 could be erroneously operated or damaged. Thus, in embodiments, the sterilization unit 700 is disposed in the front upper portion of the tub front cover 300.

In that case, the sterilization unit 700 is disposed closer to the fan 810 than the hot air supply duct 820 of the drying device 800. In other words, the distance between the sterilization unit 700 and the fan 810 is shorter than the distance between the sterilization unit 700 and the hot air supply duct 820. In this regard, the distance between certain two members refers to the minimum distance between the two members.

When the drying device 800 is operated, the air passing through the fan 810 of the drying device 800 is moved along the hot air supply duct 820 and is heated by the drying heater 830. Thereafter, the air passing through the tub 200 and the drum 400 is cooled and dehumidified by condensed water while moving along the hot air discharge duct 840. Thus, the temperature of the air introduced into the fan 810 may be lowest among the temperatures of the entire air circulation line of the drying device 800. As a result, in embodiments, the wall-mounted washing machine 10 prevents overheating of the sterilization unit 700 by disposing the sterilization unit 700 adjacent to the fan 810.

The drying device 800 supplies hot air into the tub 200 to dry washing objects. The drying device 800 may include a fan 810 coupled to the upper portion of the tub 200 and configured to circulate air, a hot air supply duct 820 connected to the fan 810 and configured to supply the air to the tub 200 through a hot air introduction port formed in the tub front cover 300, a drying heater 830 provided inside the hot air supply duct 820 and configured to heat the air introduced into the hot air supply duct 820, and a hot air discharge duct 840 connected to the tub 200 and configured to guide the air passing through the tub 200 toward the fan 810. In the drying device 800, the fan 810, the hot air supply duct 820, the tub 200, and the hot air discharge duct 840 may form an air circulation path. In other words, the drying device 800 may perform drying work by circulating the air existing inside the wall-mounted washing machine 10.

A method of drying the washing objects with the drying device 800 will now be briefly described. When the washing is completed and the drying operation is performed, the fan 810 is operated. As air is moved along the hot air supply duct 820 by the operation of the fan 810, the air is heated to a high temperature by the drying heater 830, thereby producing hot air. The hot air is then introduced into the tub 200 and the drum 400 through the tub front cover 300. The hot air makes contact with the objects inside the drum 400 to dry those objects. Thereafter, the hot air used for drying the washing objects is introduced into the hot air discharge duct 840 through the hot air discharge port formed in the rear lower portion of the tub 200. While passing through the hot air discharge duct 840, the hot air is cooled and dehumidified by the condensed water injected into the hot air discharge duct 840. In this state, the hot air may be re-introduced into the fan 810. The objects that were washed may be dried by repeatedly performing such a process.

The operations and effects of the wall-mounted washing machine 10 according to embodiments of the present disclosure will now be described.

If a washing operation is selected using the operation unit 130b of the front panel 130 after objects to be washed are placed inside the drum 400, the washing operation is performed by the wall-mounted washing machine 10. In this case, the washing operation may include a washing process, a rinsing process, a water removal process, and a drying process.

During the washing process, the rinsing process, the water removal process, and the drying process performed after the objects to be washed are put into the drum 400, the sterilization unit 700 radiates ultraviolet light on the objects present inside the drum 400 whenever such a need arises, thereby sterilizing and disinfecting those objects. In addition, even after the drying process is completed, the sterilization unit 700 may radiate ultraviolet light on the objects inside the drum 400 to sterilize and disinfect those objects. If the moisture content in the objects is low, the sterilizing power of the ultraviolet light radiated from the sterilization unit 700 is increased, reducing yellowing of the objects. Therefore, it is most effective to radiate ultraviolet light on the objects to be washed before the washing water is supplied into the drum 400, during the drying process, or after completion of the drying process.

In the case where the wall-mounted washing machine 10 performs a tub cleaning operation rather than the washing operation, the sterilization unit 700 radiates ultraviolet light into the drum 400 to remove contaminants that may remain inside the drum 400 and to sterilize and disinfect the interior of the drum 400.

From the foregoing, it will be appreciated that various embodiments of the present disclosure have been described herein for purposes of illustration, and that various modifications may be made without departing from the scope and spirit of the present disclosure. The example embodiments disclosed in the specification of the present disclosure do not limit the present disclosure. The scope of the present disclosure will be interpreted by the claims below, and it will be construed that all techniques within the scope equivalent thereto belong to the scope of the present disclosure.

What is claimed is:

1. A wall-mounted washing machine, comprising:
a cabinet;
a tub disposed in the cabinet and having an open front surface;
a tub front cover coupled to the front surface of the tub and having an opening corresponding to the open front surface of the tub;
a drum rotatably installed inside the tub;
a gasket fitted to a rim of the opening of the tub front cover; and
a sterilization unit coupled to a front surface of the tub front cover, wherein at least a part of the sterilization unit is configured to penetrate a side wall around the opening of the tub front cover,
wherein an insertion portion into which the sterilization unit is inserted is at a front portion of the gasket, and
wherein the sterilization unit is in the insertion portion in a sealed state.

2. The washing machine of claim 1, wherein the sterilization unit comprises:
a fastening portion fastened to a front surface of the tub front cover;
a case portion connected to the fastening portion and disposed in the insertion portion of the gasket;
an ultraviolet light source unit disposed inside the case portion and configured to emit ultraviolet light; and
a cover portion connected to the case portion and configured to cover the ultraviolet light source unit, wherein at least a part of the cover portion is exposed through an exposure hole in the insertion portion of the gasket.

3. The washing machine of claim 2, wherein the ultraviolet light source unit comprises:
a printed circuit board disposed inside the case portion;
a support means disposed inside the case portion and configured to support the printed circuit board; and
an ultraviolet light emitting diode mounted on the printed circuit board.

4. The washing machine of claim 2, wherein one surface of the case portion to which the cover portion is coupled is inclined toward the inside of the drum.

5. The washing machine of claim 1, wherein the sterilization unit is disposed in a front upper portion of the tub front cover.

6. The washing machine of claim 1, further comprising:
a drying device coupled to an upper outer surface of the tub and configured to dry washing objects, wherein the drying device comprises:
a fan coupled to an upper portion of the tub and configured to circulate air;
a hot air supply duct connected to the fan and configured to supply the air into the tub;
a drying heater provided inside the hot air supply duct and configured to heat the air introduced into the hot air supply duct; and
a hot air discharge duct connected to the tub and configured to guide the air passing through the tub toward the fan.

7. The washing machine of claim 6, wherein the sterilization unit is disposed closer to the fan than the hot air supply duct.

8. A wall-mounted washing machine, comprising:
a tub having an open front surface;
a tub front cover coupled to the front surface of the tub and having an opening corresponding to the open front surface of the tub;
a drum rotatably installed inside the tub;
a gasket fitted to a rim of the opening of the tub front cover; and
a sterilization unit coupled to a front surface of the tub front cover,
wherein at least a part of the sterilization unit is configured to penetrate a side wall around the opening of the tub front cover and fixed by the gasket in a sealed state.

9. The washing machine of claim 8, wherein the sterilization unit comprises:
a fastening portion fastened to a front surface of the tub front cover;
a case portion connected to the fastening portion and disposed in an insertion portion of the gasket;
an ultraviolet light source unit disposed inside the case portion and configured to emit ultraviolet light; and
a cover portion connected to the case portion and configured to cover the ultraviolet light source unit, wherein at least a part of the cover portion is exposed through an exposure hole formed in the insertion portion of the gasket.

10. The washing machine of claim 9, wherein the ultraviolet light source unit comprises:
a printed circuit board disposed inside the case portion;
a support means disposed inside the case portion and configured to support the printed circuit board; and
an ultraviolet light emitting diode mounted on the printed circuit board.

11. The washing machine of claim 9, wherein one surface of the case portion to which the cover portion is coupled is inclined toward the inside of the drum.

12. The washing machine of claim 8, wherein the sterilization unit is disposed in a front upper portion of the tub front cover.

13. The washing machine of claim 8, further comprising:
a drying device coupled to an upper outer surface of the tub and configured to dry washing objects, wherein the drying device comprises:
a fan coupled to an upper portion of the tub and configured to circulate air;
a hot air supply duct connected to the fan and configured to supply the air into the tub;
a drying heater provided inside the hot air supply duct and configured to heat the air introduced into the hot air supply duct; and
a hot air discharge duct connected to the tub and configured to guide the air passing through the tub toward the fan.

14. The washing machine of claim 13, wherein the sterilization unit is disposed closer to the fan than the hot air supply duct.
